(19) United States
(12) Patent Application Publication
(10) Pub. No.: US 2004/0014201 A1
(43) Pub. Date: Jan. 22, 2004

(54) Micro-magnetoelastic biosensor array for detection of DNA hybridization and fabrication method thereof

(75) Inventors: Chang-Kyung Kim, Seoul (KR); Chong-Seung Yoon, Seoul (KR); Ji-Hyun Lee, Seoul (KR); Cheong-Sik Yu, Seoul (KR)

Correspondence Address: MERCHANT & GOULD PC, P.O. BOX 2903, MINNEAPOLIS, MN, 55402-0903, US

(73) Assignee: HANYANG HAK WON CO., LTD., Seoul (KR)

(21) Appl. No.: 10/268,405

(22) Filed: Oct. 9, 2002

(30) Foreign Application Priority Data
Jul. 22, 2002 (KR) 2002-42879

(51) Int. Cl. C12M 1/34

(52) U.S. Cl. 435/287.2; 435/6

(57) ABSTRACT

The present invention provides a micro-magnetoelastic biosensor array for detection of the hybridization of target DNA and a method of fabricating such biosensor arrays. The biosensor array activate the magnetoelastic biosensors vibrated by an AC magnetic field, thus simply and quickly analyzing genetic materials as well as obtaining a large amount of evolving information through a real-time solution monitoring of the DNA immobilization and hybridization processes, without labeling the target sample using radioactive isotopes, enzymes or fluorescent dyes. The method of fabricating the biosensor array comprises the steps of: depositing a silicon nitride film on a lower surface of a silicon wafer, and depositing a tungsten thin film on a top surface of the silicon wafer through a sputtering technique; depositing a magnetoelastic sensor material film on a top surface of the tungsten thin film; patterning the magnetoelastic sensor material film into a predetermined shape through a photolithographic technique; depositing a gold layer for DNA immobilization on a top surface of the patterned magnetoelastic sensor material film through a sputtering technique; depositing a tungsten capping layer through a sputtering technique; patterning the deposited tungsten thin film; etching the silicon wafer in a solution; and removing the tungsten capping layer.

### BACKGROUND OF THE INVENTION

1. Field of the Invention

The present invention relates, in general, to magnetoelastic biosensor arrays for detection of DNA hybridization and a method of fabricating such biosensor arrays and, more particularly, to a micro-magnetoelastic biosensor array for detection of the hybridization of target DNA and a method of fabricating such biosensor arrays, which provide an array of micro-magnetoelastic biosensors capable of performing a quick and precise analysis of genetic materials of animals and plants.

2. Description of the Prior Art

In molecular biology, in which observation and research of biological compositions of animals and plants are performed, proteins and complex biological molecules may be analyzed through qualitative and quantitative analysis in an effort to prevent diseases, diagnose diseases, and detect/characterize viruses, bacteria and parasites, according to the determination of presence and/or concentrations of specific proteins on the basis of the analysis results.

In order to perform the screening of various target biomaterials, such as DNA, RNA or proteins, and the detection/characterization of viruses, bacteria, and parasites, nucleic acid hybridization, which relies on the complementary coupling of specific DNA fragments with target analytes, is frequently used in molecular biology. Radioactively-labeled oligonucleotide probes are typically employed to detect the hybridization of target DNA.

In addition, another method of detection of the DNA hybridization, such as a direct detection method for DNA binding, has been proposed and used in molecular biology. In such direct detection methods, DNA probes are immobilized onto solid surfaces by employing a variety of techniques, such as photochemical reaction techniques which may be referred to “A photochemical method for the manufacture of ordered arrays of oligonucleotide probes for DNA sequencing without the use of lithographic masks” (PCT Laid-open Publication No. WO 9942813 A1 19990826), lithographic techniques which may be referred to “Lithographic techniques for the fabrication of oligonucleotide arrays” (J. Photopolym. Sci. Technol., 13(4), 551-558, 2000), and “Light-directed, specially addressable parallel chemical synthesis” (Science 251, 767-773, 1991), and surface modification and direct chemical absorption techniques which may be referred to “Covalent attachment of hybridizable oligonucleotides to glass supports” (Analytical Biochemistry 247, 96-101, 1997), and “Scanning tunneling microscopy of mercapto-hexyloligonucleotides attached gold” (Biophysical Journal 71, 1079-1086, 1996).

However, the methods of detecting the hybridization of target DNA using radioactively-labeled oligonucleotide probes are problematic in that it is necessary to undesirably wait several days for the hybridization of target DNA. Another problem of the detection methods using the radioactive labels resides in that the radioactive labels are short-lived, so it is necessary to accomplish the subsequent radiographic analysis within a short period of time.

The photochemical methods of detecting the hybridization of target DNA using chemiluminophore or fluorescent dyes are problematic in that the methods are also time-consuming and require highly skilled personnel.

In the DNA detection methods using the lithographic techniques, a DNA chip including an array of micro-oligonucleotide probes is used to simultaneously monitor the hybridizations of parallel base pairs. Such a DNA chip may be referred to “Light-generated oligonucleotide arrays for rapid DNA sequence analysis” (Proc. Natl. Acad. Sci. U.S.A., 91(11), 5022-5026, 1994). However, the DNA detection methods using the lithographic techniques are problematic in that it is necessary to use expensive instruments as well as label the probes using fluorescent dyes. In recent years, CMOS biochips for the detection of DNA hybridization have been developed as disclosed in “Biochip on CMOS: an active matrix address array for DNA analysis” (Sensors & Actuators, B61, 154-162, 1999). However, the use of CMOS biochips in the detection of the DNA hybridization is problematic in that it is necessary to use precise on-board razors to detect hybridized DNA probes.

### SUMMARY OF THE INVENTION

Accordingly, the present invention has been made keeping in mind the above problems occurring in the prior art, and an object of the present invention is to provide a micro-magnetoelastic biosensor array for detection of the hybridization of target DNA and a method of fabricating such biosensor arrays, which activate the array of magnetoelastic biosensors vibrated by an AC magnetic field, thus simply and quickly analyzing genetic materials of animals and plants as well as obtaining a large amount of evolving information through a real-time solution monitoring of the DNA immobilization and hybridization processes.

In order to accomplish the above objects, the present invention provides a micro-magnetoelastic biosensor array for detection of hybridization of target DNA, comprising: a magnetoelastic biosensor to which an AC magnetic field is applied in an axial direction; and a plurality of DNA probes immobilized on a platform connected to the magnetoelastic biosensor, and hybridized with target DNA sequences, and changed in a resonant frequency thereof in response to the AC magnetic field applied to the magnetoelastic biosensor.

The present invention also provides a method of fabricating a micro-magnetoelastic biosensor array for detection of hybridization of target DNA, comprising the steps of: depositing a silicon nitride film on a lower surface of a silicon wafer, and depositing a tungsten thin film on a top surface of the silicon wafer through a sputtering technique; depositing a magnetoelastic sensor material film on a top surface of the tungsten thin film; patterning the magnetoelastic sensor material film into a predetermined shape through a photolithographic technique; depositing a gold layer for DNA immobilization on a top surface of the patterned magnetoelastic sensor material film through a sputtering technique; depositing a tungsten capping layer through a sputtering technique; patterning the deposited tungsten thin film; etching the silicon wafer in a solution; and removing the tungsten capping layer.

### BRIEF DESCRIPTION OF THE DRAWINGS

The above and other objects, features and other advantages of the is present invention will be more clearly understood from the following detailed description taken in conjunction with the accompanying drawings, in which:

FIGS. 1a and 1b are views showing the construction of a micro-magnetoelastic biosensor array in accordance with the present invention, in which FIG. 1a shows the magnetoelastic biosensor array with immobilized DNA probes, and FIG. 1b shows the magnetoelastic biosensor array with hybridized DNA probes;

FIG. 2 is a view of a system for detecting the DNA hybridization through a measurement of resonant frequency of the micro-magnetoelastic biosensor array in accordance with the present invention;

FIGS. 3a to 3h are views showing a method of fabricating the micro-magnetoelastic biosensor array in accordance with the present invention;

FIG. 4 is a view showing a formation of micro-magnetoelastic biosensors by patterning the biosensors on a single array in accordance with an embodiment of the present invention; and

FIG. 5 is a view showing a formation of micro-magnetoelastic biosensors by patterning the biosensors on multiple arrays in accordance with another embodiment of the present invention.

### DETAILED DESCRIPTION OF THE INVENTION

Reference should now be made to the drawings, in which the same reference numerals are used throughout the different drawings to designate the same or similar components.

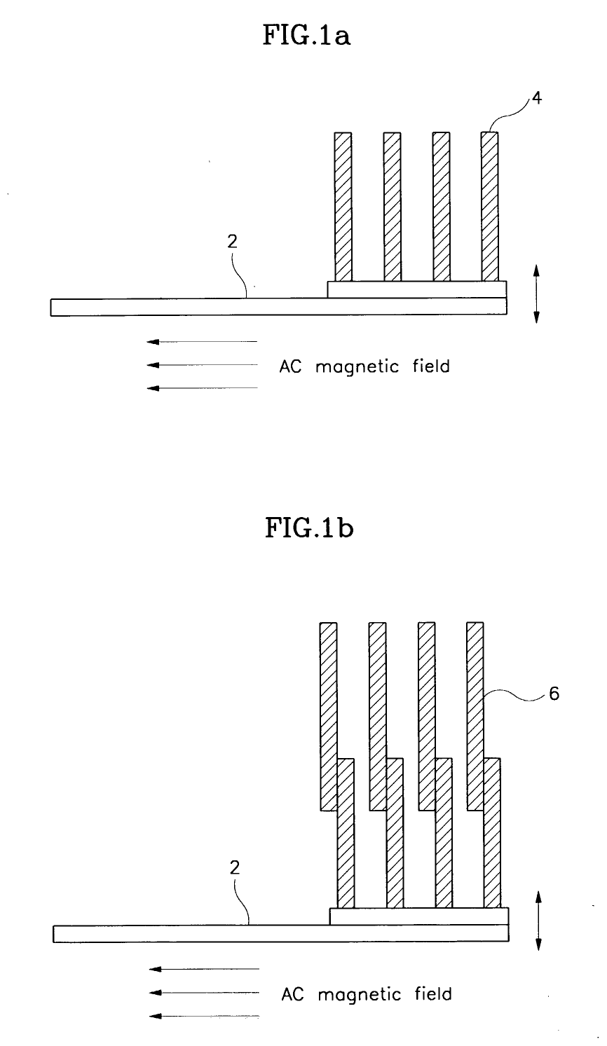

FIGS. 1a and 1b are views showing the construction of a micro-magnetoelastic biosensor array in accordance with the present invention, in which FIG. 1a shows the magnetoelastic biosensor array with immobilized DNA probes, and FIG. 1b shows the magnetoelastic biosensor array with hybridized DNA probes.

As shown in FIG. 1a, the micro-magnetoelastic biosensor array in accordance with the present invention is fabricated such that each set of magnetoelastic biosensors 2 has a different geometrical configuration in order to impart each set of biosensors 2 with a unique harmonic resonant frequency. Different DNA probes 4 are immobilized on the surface of the magnetoelastic biosensors array. In the present invention, the magnetoelastic thin film is used as a platform on which the DNA probes 4 are immobilized.

The hybridization of target DNA with the DNA probes 4 is monitored by a change in physical properties of the probe platform, such as the number of resonant vibrations and an index of refraction. Therefore, it is possible to quickly and simply analyze genetic materials as well as obtain a large amount of evolving information through a real-time solution monitoring of the DNA immobilization and hybridization processes.

The DNA probes 4 are vibrated at a predetermined harmonic resonant frequency relative to an AC magnetic field applied to each set of magnetoelastic biosensors 2 in an axial direction of the set of biosensors 2. The hybridization of target DNA with the DNA probes 4 is promoted at the unique harmonic resonant frequency of each set of magnetoelastic biosensors 2, and the array of magnetoelastic biosensors 2 is activated by applying a time-varying magnetic field at the predetermined harmonic resonant frequency of each set of magnetoelastic biosensors 2 to assist the sample agitation and expedite the hybridization with the DNA probes 4.

When performing DNA hybridization with the DNA probes 4, it is possible to monitor the hybridization of target DNA by a change in physical properties of the probe platform, such as the number of resonant vibrations and an index of refraction. Therefore, it is possible to quickly and simply analyze genetic materials as well as obtain a large amount of evolving information through a real-time solution monitoring of the DNA immobilization and hybridization processes. Such an advantage of the present invention is the same as expected from conventional direct detection of target DNA.

The array of magnetoelastic biosensors 2 according to the present invention, which is used to detect the hybridization of the DNA probes by the use of a pick-up coil, is based on Co—Fe amorphous alloy with high magnetostriction coefficient (Metall. Mater. Trans. 27A, 3203-3213, 1996, and U.S. Pat. No. 6,057,766). The above magneto elastic sensors basically rely on the mechanical vibration induced by a magnetic field impulse. The resonant frequency of such magnetoelastic sensors is changed in response to different environmental parameters, such as temperature, pressure, fluid flow velocity and mass loading (Smart Mater. Struct., 10(2), 347-353, 2001). Using the above property, thin film magnetoelastic sensors can remotely measure the temperature and pressure (Smart Mater. Struct., 8(5), 639-646, 1999).

As shown in FIG. 1b, DNA probes 6 hybridized with target biomaterials are fixedly arrayed on each set of magnetoelastic sensors 2. In the array of magnetoelastic sensors 2, the resonant frequency of each set of magnetoelastic sensors 2 is measured by the hybridized DNA probes 6 which detect a change in the resonant frequency of the sensors 6 caused by a micro-change in the mass and direction of the probe platform. That is, the DNA probes 6 hybridized with target DNA sequences measure the change in the unique hybridization frequency.

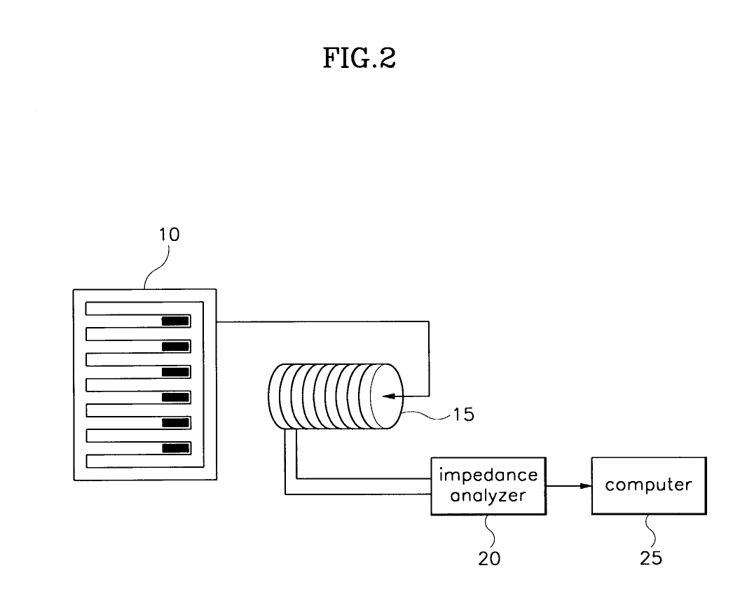

FIG. 2 is a view of a system for detecting the DNA hybridization through a measurement of resonant frequency of the micro-magnetoelastic biosensor array in accordance with the present invention.

As shown in FIG. 2, the system for detecting the DNA hybridization using the magnetoelastic biosensor array of the present invention includes a micro-sensing coil 15, an impedance analyzer 20 and a computer 25, in addition to the magnetoelastic biosensor array 10.

The magnetoelastic biosensor array 10 is fabricated with an array of magnetoelastic biosensors 2. In the biosensor array 10, the resonant frequency of each sensor 2 is monitored through the micro-sensing coil 15 by detecting a change in impedance of the sensing coil 15 while the frequency of the AC magnetic field is scanned through a specific range.

The sensing coil 15 senses the change in impedance caused by a change in the resonant frequency of the biosensor array 10 sensing coil 15, and generates impedance value signals.

The impedance analyzer 20 excites the sensing coil 15 at a given frequency range of 1 MHz-500 MHz preset relative to the resonant frequency of the magnetoelastic biosensor array 10, and measures the impedance of the sensing coil 15 at each frequency increment. A unique resonant frequency of the biosensor array 10 is measured at the peak response of the measured impedance value.

The biosensor array 10 is thus hybridized with the target DNA sequence while the biosensors 2 are excited to enhance the base pairs of the DNA probes 6. The hybridization is detected by repeating the resonant frequency measurement, and scanning through the frequency range.

The computer 25 controls the detecting system to display the process of hybridization of the DNA probes with the target DNA sequence at the peak response of the measured impedance value according to the resonant frequency of the biosensor array 10, which is measured and analyzed by the impedance analyzer 20.

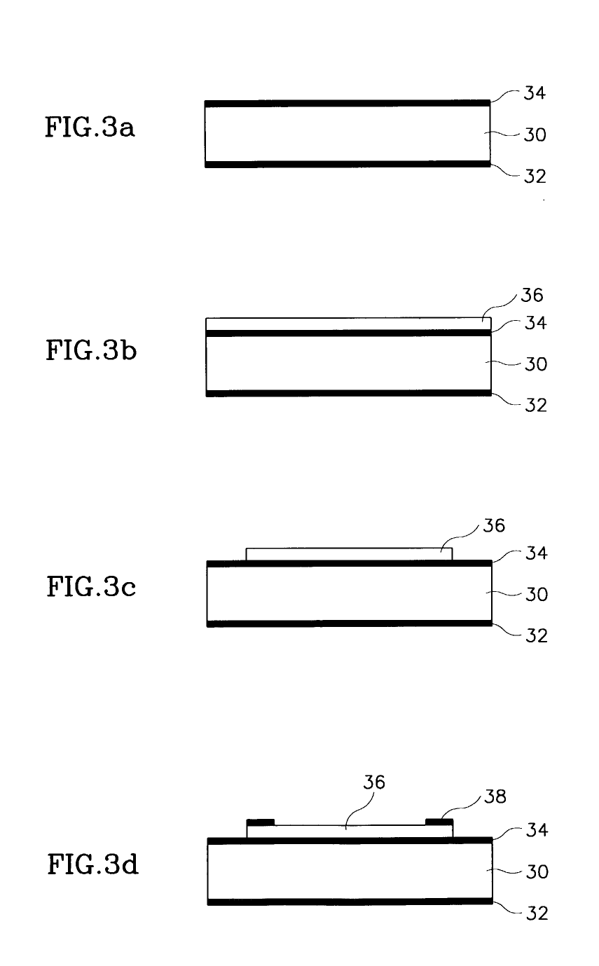

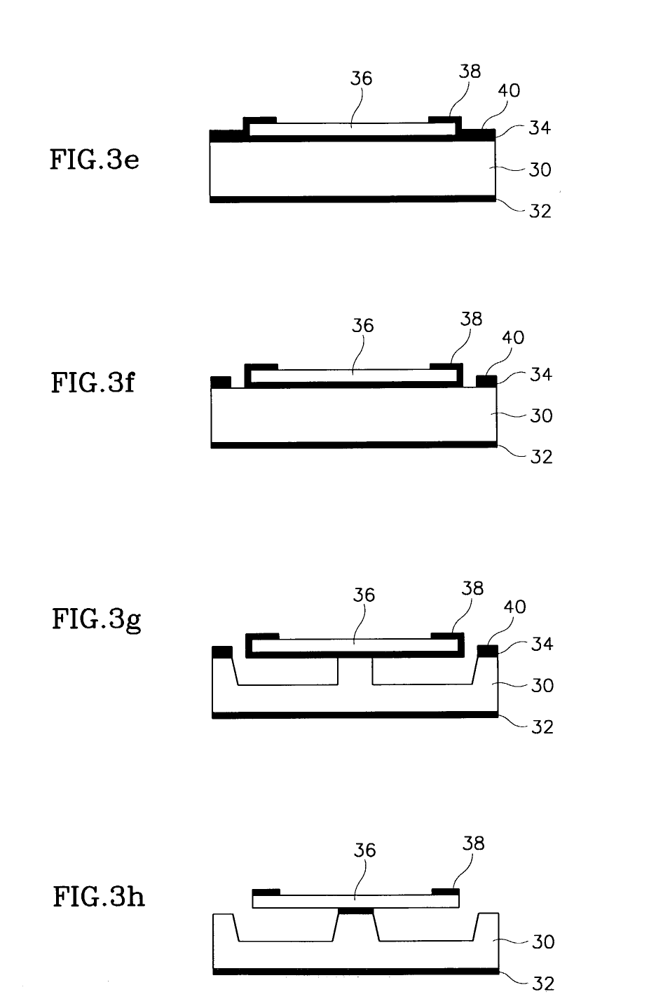

The method of fabricating the micro-magnetoelastic biosensor array in accordance with the present invention will be described herein below with reference to FIGS. 3a to 3h.

As shown in FIG. 3a, a silicon nitride (SiNₓ) film 32 of 500-2000 nm thickness is primarily deposited on the lower surface of a silicon wafer 30 to protect the biosensor during an etching process using ECR-plasma CVD. A tungsten thin film 34 of 10-1000 nm thickness is secondarily deposited on the top surface of the silicon wafer 30 through an RF magnetron sputtering system (tungsten 400, 6 mtorr argon).

A magnetoelastic sensor material film 36 consisting of CoₓFe₈₀ₓ(BSi)₂₀ (20<x<60) is deposited on the top surface of the tungsten thin film 34 using the RF magnetron sputtering system, as shown in FIG. 3b. In such a case, the thickness of the magnetoelastic sensor material film 36 is 50-2000 nm.

Thereafter, the magnetoelastic sensor material film 36 is patterned into a desired shape using a standard photolithographic method, as shown in FIG. 3c. In such a case, the magnetoelastic sensor material film 36 is etched with a 3%-HNO₃ solution (etch rate: 20 nm/sec).

After the patterning of the magnetoelastic sensor material film 36, a patch of gold layer 38 for DNA immobilization is deposited on the top surface of the patterned sensor material film 36 along the edge of the patterned sensor material film 36 using a contact metal mask, as shown in FIG. 3d. The deposition of the gold layer 38 is done with the RF magnetron sputtering system to accomplish a gold layer thickness of 10-100 nm.

After the deposition of the gold layer 38, a tungsten capping layer 40 having a thickness of 10-1000 nm is deposited using the RF magnetron sputtering system, as shown in FIG. 3e.

After the deposition of the tungsten capping layer 40, the deposited tungsten thin film 34 is patterned using H₂O₂ at a temperature of 20° C. (etch rate: 30 nm/sec), as shown in FIG. 3f.

The silicon wafer 30 is, thereafter, wet-etched in a 30%-KOH solution at a temperature of 80° C. (etch rate: 2 μm/sec), thus forming a cantilever beam as shown in FIG. 3g.

After etching the silicon wafer 30 to form the cantilever beam, the tungsten capping layer 40 is removed using H₂O₂ at a temperature of 20° C., as shown in FIG. 3h.

In order to experimentally immobilize the DNA probes, an 18-mer single-stranded oligonucleotide with a sequence 5′-CAG AGG TTG AGT CCT TTG-3′ was used. The 18-mer single-stranded oligonucleotide probe was modified by introducing a dithioethoxy group to the 5′-phosphate end.

In order to attach the disuiphide group at the 5′-phosphate end, water-soluble carbodimide was used. In such a case, the gold surface was rinsed with water and ethanol prior to the immobilization. The DNA probe was directly immobilized on the gold surface by applying the 18-mer oligonucleotide probe with the disulphide group which is known to form chemical bonds with the gold surface. The sensor was immersed in a 0.3 M NaCl solution, which contained 10 μg/ml of the DNA probe with the disulphide group, for 1 hour, then rinsed.

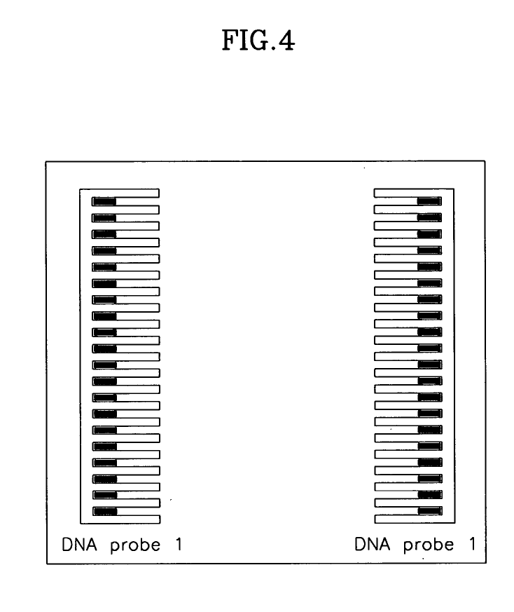

FIG. 4 is a view showing a formation of micro-magnetoelastic biosensors by patterning the biosensors on a single array in accordance with an embodiment of the present invention.

The array of magnetoelastic sensors with immobilized DNA probes was fabricated as follows. In order to generate a sufficient signal in the sensing coil, a plurality of biosensors, for example, 40 biosensors, were lithographed onto a single cell as shown in FIG. 4, and the masks were modified to pattern a multiple number of cantilever beams.

The magnetoelastic biosensor was inserted into a micro-sensing coil, which had a diameter of 3-10 mm and 50-500 turns. The impedance analyzer 20 connected to the sensing coil was used to detect the peak impedance while the coil excitation frequency was varied from 1 MHz-500 MHz.

In order to activate the biosensors to hybridize the sensors, a buffer solution was prepared with 0.05 M 4-(2-hydroxyethyl)-1-piperazine-ethanesulfonic acid and 0.2 M NaCl at pH 7.5, and the buffer solution was applied to the DNA probe. In such a case, the resonant frequency was recalibrated in order to compensate for the damping effect of the solution. 1-100 μl of aqueous solution containing the target DNA was introduced.

After introduction of the target DNA solution, the biosensor was activated at the resonant frequency for 30 minutes in order to enhance the hybridization process.

After 30 minutes, the target solution was washed off. The resonant frequency measurement was repeated in order to detect the hybridization of the DNA probe. It was found that there was a detectable shift in the resonant frequency when the hybridization tests were repeated 10 times. Meanwhile, the biosensor may be also tested without the hybridization process, whose resonant frequency is reproduced reliably within 0.1%.

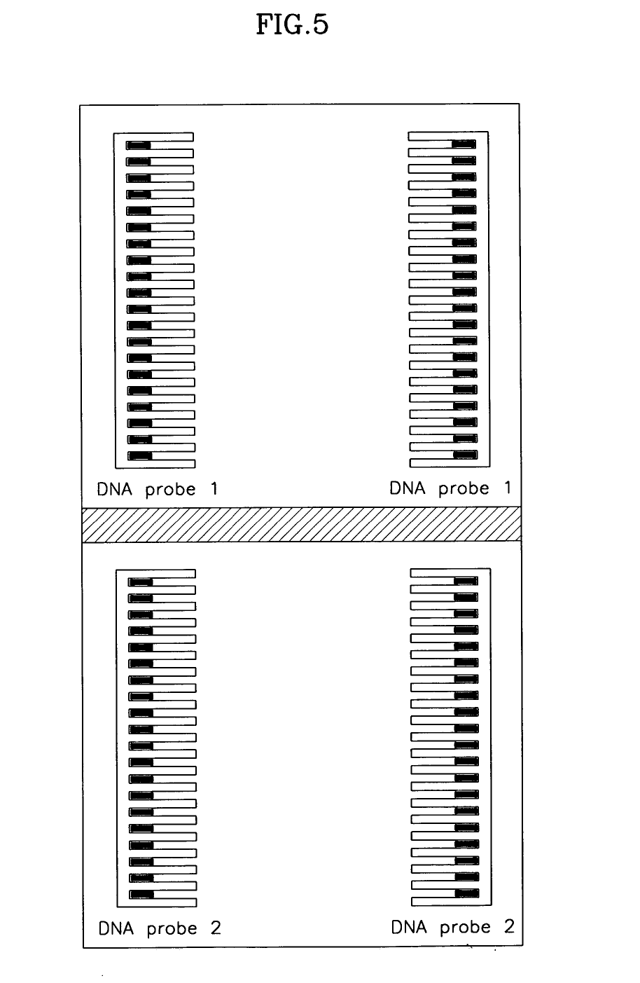

FIG. 5 is a view showing a formation of micro-magnetoelastic biosensors by patterning the biosensors on multiple arrays in accordance with another embodiment of the present invention.

As shown in FIG. 5, the present invention can be extended so that the sensor has multiple compartments containing a different set of DNA probes. Each compartment contains a set of biosensors with a unique resonant frequency, which is achieved by lithographing the sensors to different beam length and width or different geometric shape. The biosensors are separated by a compartment wall in order to prevent undesired mixing of the solution during the immobilization of the DNA probes.

Because the resonant frequency is inversely proportional to the length of the cantilever sensor, different cantilever beam lengths produce a different set of resonant frequencies. Since the resonant frequencies are discrete within the detectable limit of the impedance analyzer, the number of compartments can can be increased, each immobilized with different DNA probes and tagged with a unique resonant frequency.

As described above, the present invention provides a micro-magnetoelastic biosensor array for detection of the hybridization of target DNA, and a method of fabricating such biosensor arrays. The micro-magnetoelastic biosensor array according to the present invention detects the hybridization of target DNA by measuring the unique harmonic resonant frequency of a biosensor with DNA probes immobilized on a magneto-mechanically coupled amorphous metal thin film. It is thus possible to avoid the time consumption for removing uncoupled probes which are not changed in the harmonic resonant frequency. In addition, the micro-magnetoelastic biosensor array according to the present invention does not require the process of labeling a target sample with fluorescent dyes. The biosensor array thus simply, quickly and precisely analyzes genetic materials of animals and plants, as well as obtaining a large amount of evolving information through a real-time solution monitoring of the DNA immobilization and hybridization processes, at low cost.

Although a preferred embodiment of the present invention has been described for illustrative purposes, those skilled in the art will appreciate that various modifications, additions and substitutions are possible, without departing from the scope and spirit of the invention as disclosed in the accompanying claims. For example, in the preferred embodiment of the present invention, the magnetoelastic sensor has a cantilever beam shape, but it should be understood that the magnetoelastic sensor may be formed as a perforated cantilever beam type, folding type, or a coil type in accordance with the use of biosensors or the characteristics of systems using the biosensors, without affecting the functioning of the present invention.

What is claimed is:

1. A micro-magnetoelastic biosensor array for detection of hybridization of target DNA, comprising:
a magnetoelastic biosensor to which an AC magnetic field is applied in an axial direction; and
a plurality of DNA probes immobilized on a platform connected to the magnetoelastic biosensor, and hybridized with target DNA sequences, and changed in a resonant frequency thereof in response to the AC magnetic field applied to the magnetoelastic biosensor.

2. The micro-magnetoelastic biosensor array according to claim 1, wherein the change in the resonant frequency of the DNA probes caused by the hybridization of the DNA probes is sensed by a sensing coil in the formed of a change in impedance, and an impedance analyzer measures a unique resonant frequency of the biosensor at a peak response of the sensed impedance at a predetermined frequency range.

3. The micro-magnetoelastic biosensor array according to claim 2, wherein said magnetoelastic biosensor is constructed to be inserted into the sensing coil.

4. The micro-magnetoelastic biosensor array according to claim 1, wherein the magnetoelastic biosensor with the DNA probes is fabricated by patterning a plurality of biosensors on a single array.

5. The micro-magnetoelastic biosensor array according to claim 1, wherein the magnetoelastic biosensor with the DNA probes is fabricated such that the biosensor has multiple compartments containing a different set of DNA probes, each of said compartments containing a set of biosensors with a unique resonant frequency and unique length and width, said biosensors being separated by a compartment wall.

6. A method of fabricating a micro-magnetoelastic biosensor array for detection of hybridization of target DNA, comprising the steps of:
depositing a silicon nitride film on a lower surface of a silicon wafer, and depositing a tungsten thin film on a top surface of said silicon wafer through a sputtering technique;
depositing a magnetoelastic sensor material film on a top surface of said tungsten thin film;
patterning the magnetoelastic sensor material film into a predetermined shape through a photolithographic technique;
depositing a gold layer for DNA immobilization on a top surface of the patterned magnetoelastic sensor material film through a sputtering technique;
depositing a tungsten capping layer through a sputtering technique;
patterning the deposited tungsten thin film;
etching the silicon wafer in a solution; and
removing the tungsten capping layer.

7. The method according to claim 6, wherein said magnetoelastic sensor material film is a cobalt-iron non-crystal metal thin film (CoₓFe₈₀₋ₓ(BSi)₂₀ (20<x<60)).

8. The method according to claim 6, wherein said magnetoelastic sensor material film is etched in a 3%-HNO₃ solution, at the step of patterning the magnetoelastic sensor material film.

9. The method according to claim 6, wherein said deposited tungsten thin film is patterned using H₂O₂ at a temperature of 20° C., at the step of patterning the deposited tungsten thin film.

10. The method according to claim 6, wherein said silicon wafer is etched in a 30%-KOH solution at a temperature of 80° C., at the step of etching the silicon wafer in a solution.

11. The method according to claim 10, wherein at the step of etching the silicon wafer in a solution, said silicon wafer is etched to form a biosensor having a cantilever beam shape.

12. The method according to claim 6, wherein said tungsten capping layer is removed using H₂O₂ at a temperature of 20° C., at the step of removing the tungsten capping layer.
